(19) United States
(12) Patent Application Publication
(10) Pub. No.: US 2025/0021724 A1
(43) Pub. Date: Jan. 16, 2025

(54) INFORMATION PROCESSING DEVICE AND METHOD

(71) Applicant: Preferred Networks, Inc., Toyko (JP)

(72) Inventor: Ryohto SAWADA, Tokyo (JP)

(73) Assignee: Preferred Networks, Inc., Tokyo (JP)

(21) Appl. No.: 18/900,361

(22) Filed: Sep. 27, 2024

Related U.S. Application Data
(63) Continuation of application No. PCT/JP2023/013699, filed on Mar. 31, 2023.

(30) Foreign Application Priority Data
Mar. 31, 2022 (JP) 2022-060971

(51) Int. Cl. G06F 30/20

(52) U.S. Cl. CPC G06F 30/20

(57) ABSTRACT

An information processing device includes one or more memories and one or more processors. The one or more processors are configured to set a first state of a structure of a plurality of atoms to search for a reaction path from the first state up to a second state of the structure of the plurality of atoms in a chemical reaction in which the plurality of atoms are involved and search for the reaction path from the first state up to the second state by calculating, from the first state, a change in the structure of the plurality of atoms such that, in the second state, an index of mutual displacement of two or more specific atoms out of the plurality of atoms falls within a defined range.

### CROSS REFERENCE TO THE RELATED APPLICATION

This application is continuation application of International Application No. JP2023/013699, filed on Mar. 31, 2023, which claims priority to Japanese Application No. 2022-060971, filed on Mar. 31, 2022, the entire content of which are incorporated herein by reference

### FIELD

This disclosure relates to an information processing device and a method.

### BACKGROUND

The ease of a chemical reaction can be calculated from an initial state before the reaction and a final state after the reaction, and an energy difference (activation energy) in a transition state present between the initial state and the final state. As a method to find the energy in the transition state from a combination of the initial state and the final state, a NEB (Nudged Elastic Band) method or the like is known. However, it is difficult to find out an appropriate combination of the initial state and the final state regarding a complicated molecule and a molecule on a catalyst. Because of this, there has been a demand for a method capable of searching out a chemical reaction without a user giving information regarding the combination of the initial state and the final state.

For example, GRRM (registered trademark) (Global Reaction Route Mapping) uses ADDF (Anharmonic Downward Distortion Following) or the like to investigate, without any advance knowledge, to which substance an individual chemical compound easily changes (isomerizes or decomposes). The use of GRRM enables a user to find a transition state and a final state of a reaction that can occur, from information on the initial state.

A transition state can be represented as a superposition of the initial state and its normal mode vibrations. ADDF searches for the transition state by using a path where the transition state is oriented in a direction of a point where energy stabilizes most under the condition that the energy of the normal mode vibration is kept constant.

The number of transition states searchable by GRRM increases exponentially as the number of atoms increases. Therefore, regarding a large molecule, a molecule on a catalyst, or the like, it is desired to narrow down search targets to a specific reaction. For this purpose, AFIR (Artificial Force Induced Reaction) was developed as a method to automatically search for a process of a mutual reaction of a plurality of reactants by giving pseudo-attractive force or repulsive force to specific molecules or atoms so that a specific reaction easily occurs. The use of AFIR makes it possible to automatically search for a reaction that agrees with a given condition if a user gives information on the initial state and a combination of atoms or molecules that are desired to be dissociated or bonded.

However, ADDF requires calculating normal mode vibrations when finding a transition state. Further, the calculation amount of the vibration modes is proportional to the cube of the number of atoms. In the case where a first-principles calculation is used for energy calculation, there occurs no big problem because the calculation amount in many of the first-principles calculations is also proportional to the cube of the number of atoms, but in the case where a method with a less calculation amount, such as a classical potential or NNP (Neural Network Potential), is used for the energy calculation, this can constitute a bottleneck. Further, in AFIR, too weak a pseudo-potential does not cause a reaction, whereas too strong a pseudo-potential causes molecular destruction or the like even in a region other than that of a reaction that is desired to be found. This leads to a problem that parameters need to be decided for each bond that is desired to be controlled.

### BRIEF DESCRIPTION OF THE DRAWINGS

FIG. 1 is a chart schematically illustrating energies according to one embodiment.

FIG. 2 is a flowchart illustrating processing according to the embodiment.

FIG. 3 is a diagram illustrating an implementation example of an information processing device according to the embodiment.

### DETAILED DESCRIPTION

According to one embodiment, an information processing device includes one or more memories and one or more processors. The one or more processors are configured to set a first state of a structure of a plurality of atoms to search for a reaction path from the first state up to a second state of the structure of the plurality of atoms in a chemical reaction in which the plurality of atoms are involved and search for the reaction path from the first state up to the second state by calculating, from the first state, a change in the structure of the plurality of atoms such that, in the second state, an index of mutual displacement of two or more specific atoms out of the plurality of atoms falls within a defined range.

An embodiment of the present invention will be hereinafter described with reference to the drawings. The drawings and the embodiment are presented by way of example only and are not intended to limit the present invention.

In this disclosure, the information processing device predicts a reaction path by using structure optimization under a constrained displacement amount of atoms. The information processing device includes a storage circuit and a processing circuit and implements processing based on information stored in the storage circuit. This information processing device receives, from a user, an input of a pre-reaction state and a post-reaction state formed by two or more atoms to search for a stable structure that changes from the pre-reaction state up to the post-reaction state.

A difference between this method and the ADDF method will be described.

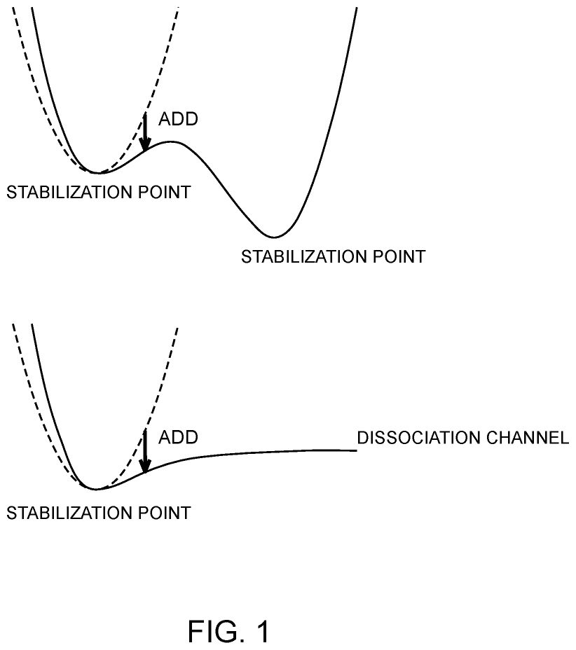

FIG. 1 is a chart schematically illustrating energies at the time of the transition from a stable state to another state. The upper chart illustrates energy at the time of the transition to another stabilization point. In the case where a molecule (or atoms, which applies to the following description of this drawing) is present at the left stabilization point, the potential of the molecule itself can be represented as a superposition of normal mode vibrations in the stable state and can be approximated by a quadric curve as indicated by the broken line.

In the case where there is another stabilization point (stabilization point on the right side) nearby, a reaction from the current stabilization point to the other stabilization point can occur. The transition from a certain stabilization point to another stabilization point is what is called a reaction. Due to the presence of the plurality of stabilization points, the potential indicated by the broken line distorts downward as illustrated in the chart. This distortion is called ADD (Anharmonic Downward Distortion). It is known that the reaction is more likely to occur as this distortion is greater.

The lower chart illustrates a potential between a stabilization point and a dissociation channel. In this case as well, the molecular potential indicated by the broken line actually distorts downward due to ADD as in the above case.

When searching for a reaction path, the processing circuit sets individual potentials as indicated by the broken line for respective molecules (stabilization points) and executes the search while determining how the potentials distort. The ADDF method is a method to search for a reaction path while searching for a direction in which ADD becomes maximum. The processing circuit searches for a transition state where ADD becomes maximum, using the assumption that the transition state is present in a direction of a point where energy stabilizes most under the condition that energy of the normal mode vibration of the molecule is kept constant.

The calculation of the normal mode vibrations requires a calculation time equal to the cube of the number of atoms and thus is difficult to apply to a large-scale system. Further, which value range should be set for an energy value of the normal mode vibration depends on the material, and thus the adjustment of hyperparameters by a user is indispensable. In this disclosure, on the other hand, the energy optimization calculation is executed under the constraint condition that the displacement amount of atoms (as an example, interatomic distance) instead of the energy of the normal mode vibration is kept at a fixed value. In the method of this disclosure, since there is no need to calculate the normal mode vibrations, the calculation cost is low. Further, a change in bond length in a chemical reaction differs little depending on the material, and thus it is possible to lower the dependence on hyperparameters.

Since the above-described method is able to search for only one transition state, there may be an additional step of giving a penalty to an already searched transition state to prevent convergence in only one transition state, thereby enabling the search for various transition states.

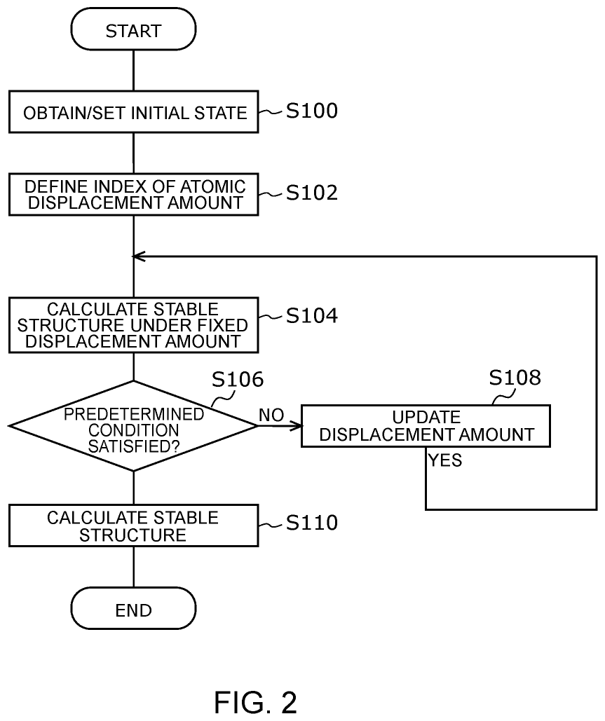

FIG. 2 is a flowchart illustrating processing according to the embodiment.

The processing circuit first obtains and sets an initial state (S100). The information processing device may include an appropriate input/output interface and obtain the initial state from a user. The initial state includes information on at least two atoms and is, for example, a structure of a molecule, atoms, or the like of a substance whose reaction path is desired to be known.

Next, the processing circuit defines an index of a displacement amount of the atoms according to a search-target reaction (S102). If the search-target reaction is, for example, a reaction such as AB->A+B, the processing circuit may set a bond length between A and B as the index. It should be noted that the index is not limited to the interatomic distance. In the case where, for example, the chemical reaction is greatly associated with a specific vibration, the processing circuit can use, as the index, the magnitude of a component of the normal mode vibration instead of the bond length.

The processing circuit calculates a stable structure while the displacement amount set as the index is kept fixed (S104). For example, the processing circuit sets the interatomic distance as the constraint condition, calculates energy values in various structures of a chemical compound such as a molecule, separately existing atoms, or the like, and searches for a structure where this energy is low. For the structure search, a known optimizer such as FIRE (Fast Inertial Relaxation Engine) or a quasi-Newton method is usable.

In the case where, for example, the interatomic distance is set as the index, the processing circuit sets the constraint condition that the distance between two given atoms is fixed at 3 [Å] or the like as the displacement amount and calculates the stable state based on the potential (energy). This distance (that is, the distance set as the constraint condition) is presented by way of a nonlimiting example only, and an appropriate range can be defined as the distance according to the situation, for example, 1 to 5 [Å] or the like.

In the case where, for example, a pre-reaction state is a state of two atoms A and B and a post-reaction state is a state in which A and B are bonded, the displacement amount as the initial value may be set to 3 [A]. By executing the optimization while giving the constraint of this fixed displacement amount, the processing circuit obtains a stable state under the condition that the distance between the atoms A and B is 3 [Å].

After obtaining the stable structure in the state where the displacement amount is fixed, the processing circuit determines whether or not the obtained stable structure satisfies a predetermined condition (S106). The predetermined condition may be, for example, a condition that the energy value in the stable structure exceeds a predetermined value, that the displacement amount does not increase/decrease even without the constraint of the displacement amount, that the displacement amount falls out of a predetermined range if the next updating of the displacement amount is executed, that the updating of the displacement amount has been done a predetermined number of times, or the like, but the predetermined condition is not limited to any of these conditions and may be a condition under which the final stable state can be appropriately obtained.

In the case where the predetermined condition is not satisfied (S106: NO), the processing circuit updates the satisfied displacement amount (S108) and iterates the process in S104. In the case where the interatomic distance is set as the index, if intending to obtain a stable structure regarding, for example, the bonding of the atoms (A+B->AB), the processing circuit updates the displacement amount so as to shorten the distance (for example, 3 [Å]->2 [Å]). Contrarily, if intending to obtain a stable structure regarding the unbonding of the atoms (AB->A+B), the processing circuit updates the displacement amount so as to elongate the distance (for example, 3 [Å]->4 [Å]). The processing circuit iterates the computation of the stable structure through the optimization in S104, with the updated displacement amount set as the fixed value.

In the case where the predetermined condition is satisfied (S106: YES), the processing circuit calculates a stable structure in an unconstrained state (S110). By executing the calculation of the stable structure in the unconstrained state after obtaining the stable structure in the constrained state, the processing circuit is capable of obtaining a quasi-stable state at a displacement destination, that is, obtaining the final state.

For example, the distance between two bonded atoms or two atoms changing to a bonded state is mostly in a very narrow region, for example, 2 to 5 [A] irrespective of the kind of atoms. Therefore, by executing the optimization under the constrained bond length, it is possible to appropriately handle not only bonded atoms but also a process up to the transition to an unbonded state or up to the transition to a bonded state.

In the above, the interatomic distance constrained within a certain range is used as the index, and this setting is effective in the case where it is desired to search for a specific chemical reaction. On the other hand, in the case where it is desired to enumerate various reactions, this displacement amount may be randomly given and thereafter fixed and the fixed amount may be used as the index.

As described above, according to this embodiment, by constraining the displacement amount, it is possible to appropriately obtain the stable state and the quasi-stable state in the state transition such as atomic bonding and molecular decomposition. As described above, data that the user needs to input is only the displacement amount which is an index of the displacement caused by a reaction, and the user need not find specific structures in the initial state and the final state. Further, in the case where the interatomic distance is set as the displacement amount, the optimization is executed while this bond length is constrained, leading to low parameter independence and making it possible to obtain a robust and stable result. Further, the complicated and high-cost computation of vibration modes or the like is not required, and it is possible to complete the processing only by iterating the simple optimization, making it possible to reduce the computation cost.

Some or all of each device (the information processing device) in the above embodiment may be configured in hardware, or information processing of software (program) executed by, for example, a CPU (Central Processing Unit), GPU (Graphics Processing Unit). In the case of the information processing of software, software that enables at least some of the functions of each device in the above embodiments may be stored in a non-volatile storage medium (non-volatile computer readable medium) such as CD-ROM (Compact Disc Read Only Memory) or USB (Universal Serial Bus) memory, and the information processing of software may be executed by loading the software into a computer. In addition, the software may also be downloaded through a communication network. Further, entire or a part of the software may be implemented in a circuit such as an ASIC (Application Specific Integrated Circuit) or FPGA (Field Programmable Gate Array), wherein the information processing of the software may be executed by hardware.

A storage medium to store the software may be a removable storage media such as an optical disk, or a fixed type storage medium such as a hard disk, or a memory. The storage medium may be provided inside the computer (a main storage device or an auxiliary storage device) or outside the computer.

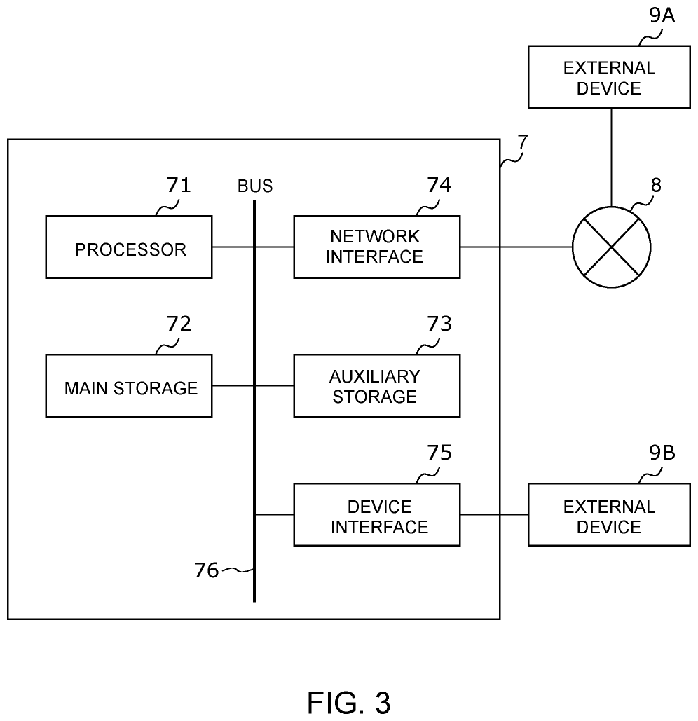

FIG. 3 is a block diagram illustrating an example of a hardware configuration of each device (the information processing device) in the above embodiments. As an example, each device may be implemented as a computer 7 provided with a processor 71, a main storage device 72, an auxiliary storage device 73, a network interface 74, and a device interface 75, which are connected via a bus 76.

The computer 7 of FIG. 3 is provided with each component one by one but may be provided with a plurality of the same components. Although one computer 7 is illustrated in FIG. 3, the software may be installed on a plurality of computers, and each of the plurality of computer may execute the same or a different part of the software processing. In this case, it may be in a form of distributed computing where each of the computers communicates with each of the computers through, for example, the network interface 74 to execute the processing. That is, each device (the information processing device) in the above embodiments may be configured as a system where one or more computers execute the instructions stored in one or more storages to enable functions. Each device may be configured such that the information transmitted from a terminal is processed by one or more computers provided on a cloud and results of the processing are transmitted to the terminal.

Various arithmetic operations of each device (the information processing device) in the above embodiments may be executed in parallel processing using one or more processors or using a plurality of computers over a network. The various arithmetic operations may be allocated to a plurality of arithmetic cores in the processor and executed in parallel processing. Some or all the processes, means, or the like of the present disclosure may be implemented by at least one of the processors or the storage devices provided on a cloud that can communicate with the computer 7 via a network. Thus, each device in the above embodiments may be in a form of parallel computing by one or more computers.

The processor 71 may be an electronic circuit (such as, for example, a processor, processing circuity, processing circuitry, CPU, GPU, FPGA, or ASIC) that executes at least controlling the computer or arithmetic calculations. The processor 71 may also be, for example, a general-purpose processing circuit, a dedicated processing circuit designed to perform specific operations, or a semiconductor device which includes both the general-purpose processing circuit and the dedicated processing circuit. Further, the processor 71 may also include, for example, an optical circuit or an arithmetic function based on quantum computing.

The processor 71 may execute an arithmetic processing based on data and/or a software input from, for example, each device of the internal configuration of the computer 7, and may output an arithmetic result and a control signal, for example, to each device. The processor 71 may control each component of the computer 7 by executing, for example, an OS (Operating System), or an application of the computer 7.

Each device (the information processing device) in the above embodiments may be enabled by one or more processors 71. The processor 71 may refer to one or more electronic circuits located on one chip, or one or more electronic circuitries arranged on two or more chips or devices. In the case of a plurality of electronic circuitries is used, each electronic circuit may communicate by wired or wireless.

The main storage device 72 may store, for example, instructions to be executed by the processor 71 or various data, and the information stored in the main storage device 72 may be read out by the processor 71. The auxiliary storage device 73 is a storage device other than the main storage device 72. These storage devices shall mean any electronic component capable of storing electronic information and may be a semiconductor memory. The semiconductor memory may be either a volatile or non-volatile memory. The storage device for storing various data or the like in each device (the information processing device) in the above embodiments may be enabled by the main storage device 72 or the auxiliary storage device 73 or may be implemented by a built-in memory built into the processor 71. For example, the storages in the above embodiments may be implemented in the main storage device 72 or the auxiliary storage device 73.

In the case of each device (the information processing device) in the above embodiments is configured by at least one storage device (memory) and at least one e processor connected/coupled to/with this at least one storage device, the at least processor may be connected to a single storage device. Or the at least storage may be connected to a single processor. Or each device may include a configuration where at least one of the plurality of processors is connected to at least one of the plurality of storage devices. Further, this configuration may be implemented by a storage device and a processor included in a plurality of computers. Moreover, each device may include a configuration where a storage device is integrated with a processor (for example, a cache memory including an L1 cache or an L2 cache).

The network interface 74 is an interface for connecting to a communication network 8 by wireless or wired. The network interface 74 may be an appropriate interface such as an interface compatible with existing communication standards. With the network interface 74, information may be exchanged with an external device 9A connected via the communication network 8. Note that the communication network 8 may be, for example, configured as WAN (Wide Area Network), LAN (Local Area Network), or PAN (Personal Area Network), or a combination of thereof, and may be such that information can be exchanged between the computer 7 and the external device 9A. The internet is an example of WAN, IEEE802.11 or Ethernet (registered trademark) is an example of LAN, and Bluetooth (registered trademark) or NFC (Near Field Communication) is an example of PAN.

The device interface 75 is an interface such as, for example, a USB that directly connects to the external device 9B.

The external device 9A is a device connected to the computer 7 via a network. The external device 9B is a device directly connected to the computer 7.

The external device 9A or the external device 9B may be, as an example, an input device. The input device is, for example, a device such as a camera, a microphone, a motion capture, at least one of various sensors, a keyboard, a mouse, or a touch panel, and gives the acquired information to the computer 7. Further, it may be a device including an input unit such as a personal computer, a tablet terminal, or a smartphone, which may have an input unit, a memory, and a processor.

The external device 9A or the external device 9B may be, as an example, an output device. The output device may be, for example, a display device such as, for example, an LCD (Liquid Crystal Display), or an organic EL (Electro Luminescence) panel, or a speaker which outputs audio. Moreover, it may be a device including an output unit such as, for example, a personal computer, a tablet terminal, or a smartphone, which may have an output unit, a memory, and a processor.

Further, the external device 9A or the external device 9B may be a storage device (memory). The external device 9A may be, for example, a network storage device, and the external device 9B may be, for example, an HDD storage.

Furthermore, the external device 9A or the external device 9B may be a device that has at least one function of the configuration element of each device (the information processing device) in the above embodiments. That is, the computer 7 may transmit a part of or all of processing results to the external device 9A or the external device 9B, or receive a part of or all of processing results from the external device 9A or the external device 9B.

In the present specification (including the claims), the representation (including similar expressions) of “at least one of a, b, and c” or “at least one of a, b, or c” includes any combinations of a, b, c, a-b, a-c, b-c, and a-b-c. It also covers combinations with multiple instances of any element such as, for example, a-a, a-b-b, or a-a-b-b-c-c. It further covers, for example, adding another element d beyond a, b, and/or c, such that a-b-c-d.

In the present specification (including the claims), the expressions such as, for example, “data as input,” “using data,” “based on data,” “according to data,” or “in accordance with data” (including similar expressions) are used, unless otherwise specified, this includes cases where data itself is used, or the cases where data is processed in some ways (for example, noise added data, normalized data, feature quantities extracted from the data, or intermediate representation of the data) are used. When it is stated that some results can be obtained “by inputting data,” “by using data,” “based on data,” “according to data,” “in accordance with data” (including similar expressions), unless otherwise specified, this may include cases where the result is obtained based only on the data, and may also include cases where the result is obtained by being affected factors, conditions, and/or states, or the like by other data than the data. When it is stated that “output/outputting data” (including similar expressions), unless otherwise specified, this also includes cases where the data itself is used as output, or the cases where the data is processed in some ways (for example, the data added noise, the data normalized, feature quantity extracted from the data, or intermediate representation of the data) is used as the output.

In the present specification (including the claims), when the terms such as “connected (connection)” and “coupled (coupling)” are used, they are intended as non-limiting terms that include any of “direct connection/coupling,” “indirect connection/coupling,” “electrical connection/coupling,” “communicative connection/coupling,” “operative connection/coupling,” “physical connection/coupling,” or the like. The terms should be interpreted accordingly, depending on the context in which they are used, but any forms of connection/coupling that are not intentionally or naturally excluded should be construed as included in the terms and interpreted in a non-exclusive manner.

In the present specification (including the claims), when the expression such as “A configured to B,” this may include that a physically structure of A has a configuration that can execute operation B, as well as a permanent or a temporary setting/configuration of element A is configured/set to actually execute operation B. For example, when the element A is a general-purpose processor, the processor may have a hardware configuration capable of executing the operation B and may be configured to actually execute the operation B by setting the permanent or the temporary program (instructions). Moreover, when the element A is a dedicated processor, a dedicated arithmetic circuit, or the like, a circuit structure of the processor or the like may be implemented to actually execute the operation B, irrespective of whether or not control instructions and data are actually attached thereto.

In the present specification (including the claims), when a term referring to inclusion or possession (for example, “comprising/including,” “having,” or the like) is used, it is intended as an open-ended term, including the case of inclusion or possession an object other than the object indicated by the object of the term. If the object of these terms implying inclusion or possession is an expression that does not specify a quantity or suggests a singular number (an expression with a or an article), the expression should be construed as not being limited to a specific number.

In the present specification (including the claims), although when the expression such as “one or more,” “at least one,” or the like is used in some places, and the expression that does not specify a quantity or suggests a singular number (the expression with a or an article) is used elsewhere, it is not intended that this expression means “one.” In general, the expression that does not specify a quantity or suggests a singular number (the expression with a or an as article) should be interpreted as not necessarily limited to a specific number.

In the present specification, when it is stated that a particular configuration of an example results in a particular effect (advantage/result), unless there are some other reasons, it should be understood that the effect is also obtained for one or more other embodiments having the configuration. However, it should be understood that the presence or absence of such an effect generally depends on various factors, conditions, and/or states, etc., and that such an effect is not always achieved by the configuration. The effect is merely achieved by the configuration in the embodiments when various factors, conditions, and/or states, etc., are met, but the effect is not always obtained in the claimed invention that defines the configuration or a similar configuration.

In the present specification (including the claims), when the term such as “maximize/maximization” is used, this includes finding a global maximum value, finding an approximate value of the global maximum value, finding a local maximum value, and finding an approximate value of the local maximum value, should be interpreted as appropriate accordingly depending on the context in which the term is used. It also includes finding on the approximated value of these maximum values probabilistically or heuristically. Similarly, when the term such as “minimize/minimization” is used, this includes finding a global minimum value, finding an approximated value of the global minimum value, finding a local minimum value, and finding an approximated value of the local minimum value, and should be interpreted as appropriate accordingly depending on the context in which the term is used. It also includes finding the approximated value of these minimum values probabilistically or heuristically. Similarly, when the term such as “optimize/optimization” is used, this includes finding a global optimum value, finding an approximated value of the global optimum value, finding a local optimum value, and finding an approximated value of the local optimum value, and should be interpreted as appropriate accordingly depending on the context in which the term is used. It also includes finding the approximated value of these optimal values probabilistically or heuristically.

In the present specification (including claims), when a plurality of hardware performs a predetermined process, the respective hardware may cooperate to perform the predetermined process, or some hardware may perform all the predetermined process. Further, a part of the hardware may perform a part of the predetermined process, and the other hardware may perform the rest of the predetermined process. In the present specification (including claims), when an expression (including similar expressions) such as “one or more hardware perform a first process and the one or more hardware perform a second process,” or the like, is used, the hardware that perform the first process and the hardware that perform the second process may be the same hardware, or may be the different hardware. That is: the hardware that perform the first process and the hardware that perform the second process may be included in the one or more hardware. Note that, the hardware may include an electronic circuit, a device including the electronic circuit, or the like.

In the present specification (including the claims), when a plurality of storage devices (memories) store data, an individual storage device among the plurality of storage devices may store only a part of the data or may store the entire data. Further, some storage devices among the plurality of storage devices may include a configuration for storing data.

While certain embodiments of the present disclosure have been described in detail above, the present disclosure is not limited to the individual embodiments described above. Various additions, changes, substitutions, partial deletions, etc. are possible to the extent that they do not deviate from the conceptual idea and purpose of the present disclosure derived from the contents specified in the claims and their equivalents. For example, when numerical values or mathematical formulas are used in the description in the above-described embodiments, they are shown for illustrative purposes only and do not limit the scope of the present disclosure. Further, the order of each operation shown in the embodiments is also an example, and does not limit the scope of the present disclosure.

1. An information processing device comprising:
one or more memories; and
one or more processors configured to
set a first state of a structure of a plurality of atoms to search for a reaction path from the first state up to a second state of the structure of the plurality of atoms in a chemical reaction in which the plurality of atoms are involved; and
search for the reaction path from the first state up to the second state by calculating, from the first state, a change in the structure of the plurality of atoms such that, in the second state, an index of mutual displacement of two or more specific atoms out of the plurality of atoms falls within a defined range.

2. The information processing device according to claim 1,
wherein the one or more processors are configured to:
receive, from a user, information to define the range where the index of the mutual displacement of the two more specific atoms is to fall; and
search for the reaction path by calculating, from the first state, the change in the structure of the plurality of atoms based on the received information such that, in the second state, the index of the mutual displacement of the two or more specific atoms falls within the defined range.

3. The information processing device according to claim 1,
wherein the one or more processors are configured to:
set a condition expressed by the defined range, regarding the mutual displacement of the two or more specific atoms; and
search for the reaction path of the chemical reaction by iteratively calculating the change in the structure of the plurality of atoms under the condition.

4. The information processing device according to claim 3,
wherein the iterative calculation of the change in the structure of the plurality of atoms is executed by optimization calculation that is based on energy of the plurality atoms, under the condition.

5. The information processing device according to claim 1,
wherein the two or more specific atoms are two specific atoms out of the plurality of atoms, and
wherein the defined range is a range of an interatomic distance between the two specific atoms.

6. The information processing device according to claim 1,
wherein an amount of the mutual displacement of the two or more specific atoms is a displacement amount indicating a positional relationship of the two or more specific atoms, and
wherein the defined range is a range of the positional relationship of the two or more specific atoms.

7. The information processing device according to claim 6,
wherein the displacement amount indicating the positional relationship of the two or more specific atoms is a distance between the two or more specific atoms, and
wherein the defined range is a range of the interatomic distance between the two or more specific atoms.

8. The information processing device according to claim 1,
wherein the first state is an initial state in the chemical reaction.

9. The information processing device according to claim 8,
wherein the second state is a final state in the chemical reaction.

10. The information processing device according to claim 1,
wherein the defined range where the index of the mutual displacement of the two or more specific atoms is to fall has at least one limit.

11. The information processing device according to claim 1,
wherein the defined range where the index of the mutual displacement of the two or more specific atoms is to fall is not one value.

12. The information processing device according to claim 1,
wherein the defined range where the index of the mutual displacement of the two or more specific atoms is to fall is a range defined by an upper limit and a lower limit.

13. A method of searching for a reaction path, the method comprising:
setting a first state of a structure of the plurality of atoms to search for the reaction path from the first state up to a second state of the structure of the plurality of atoms in a chemical reaction in which the plurality of atoms are involved; and
searching for the reaction path from the first state up to the second state by calculating, from the first state, a change in the structure of the plurality of atoms such that, in the second state, an index of mutual displacement of two or more specific atoms out of the plurality of atoms falls within a defined range.

14. The method according to claim 13, further comprising
receiving, from the user, information to define the range where the index of the mutual displacement of the two more specific atoms is to fall,
wherein the search for the reaction path is executed by calculating, from the first state, the change in the structure of the plurality of atoms based on the received information such that, in the second state, the index of the mutual displacement of the two or more specific atoms falls within the range.

15. The method according to claim 13, further comprising
setting a condition expressed by the defined range, regarding the mutual displacement of the two or more specific atoms,
wherein the search for the reaction path is executed by iteratively calculating the change in the structure of the plurality of atoms under the condition.

16. The method according to claim 13,
wherein the two or more specific atoms are two specific atoms out of the plurality of atoms, and
wherein the defined range is a range of an interatomic distance between the two specific atoms.

17. The method according to claim 13,
wherein an amount of the mutual displacement of the two or more specific atoms is a displacement amount indicating a positional relationship of the two or more specific atoms, and
wherein the defined range is a range of the positional relationship of the two or more specific atoms.

18. The method according to claim 17,
wherein the displacement amount indicating the positional relationship of the two or more specific atoms is a distance between the two or more specific atoms, and
wherein the defined range is a range of the interatomic distance between the two or more specific atoms.

19. The method according to claim 13,
wherein the first state is an initial state in the chemical reaction, and
wherein the second state is a final state in the chemical reaction.

20. An information processing device which causes execution of a method of searching for a reaction path from a first state of a structure of a plurality of atoms up to a second state of the structure of the plurality of atoms in a chemical reaction in which the plurality of atoms are involved,
the information processing device comprising:
one or more memories; and
one or more processors configured to:
cause to calculate, from the first state, a change in a structure of the plurality of atoms such that, in the second state, an index of mutual displacement of two or more specific atoms out of the plurality of atoms falls within a defined range; and
obtain the reaction path from the first state up to the second state which path is found by the calculation.
